(19) United States
(12) Patent Application Publication
(10) Pub. No.: US 2010/0291182 A1
(43) Pub. Date: Nov. 18, 2010

(54) Drug-Loaded Fibers

(75) Inventors: Maria Palasis, Wellesley, MA (US); Upma Sharma, Somerville, MA (US); Quyhn Pham, Waltham (VN); John Marini, Weymouth, MA (US)

Correspondence Address: BINGHAM MCCUTCHEN LLP, 2020 K Street, N.W., Intellectual Property Department, WASHINGTON, DC, 20006, US

(73) Assignee: Arsenal Medical, Inc., Watertown, MA (US)

(21) Appl. No.: 12/620,334

(22) Filed: Nov. 17, 2009

Related U.S. Application Data
(60) Provisional application No. 61/146,060, filed on Jan. 21, 2009.

(51) Int. Cl. A61F 2/00; A61K 31/575; A61K 31/4535; A61K 31/4468; A61K 31/485; A61K 31/445; A61K 38/17; A61P 19/02; A61P 27/02; A61P 27/06; A61P 27/12; A61P 29/00; A61P 25/00; A61P 25/16; A61P 25/28

(52) U.S. Cl. 424/426; 424/423; 514/179; 514/326; 514/329; 514/282; 514/330; 514/18.3

(57) ABSTRACT

Implants and methods for the delivery of a therapeutic agent to a target location within a patient's body are disclosed. The implants include a fiber comprising a polymeric material and having a diameter of up to about twenty microns, and a first therapeutic agent within the fiber. The therapeutic agent is substantially in particulate form. The implants are of a variety of configurations, such as individual fibers, yarns, ropes, tubes, and patches.

### RELATED APPLICATIONS

This application claims the benefit of U.S. Provisional Application No. 61/146,060, entitled “Compositions and Methods for Treating Joint Conditions” by Palasis, et al., the disclosure of which is incorporated herein by reference.

### FIELD OF THE INVENTION

The present invention relates to drug-loaded fibers, and more specifically, to small fibers that are used to deliver drugs to target locations within a patient's body.

### BACKGROUND

Fibers have been proposed for a number of medical applications, including for the localized delivery of therapeutic agents within a patient's body. To facilitate such use, polymeric fibers are loaded with drugs and subsequently implanted within a patient to allow for the delivery of the drug over an extended period of time. The manufacture and practical application of such fibers, however, has been limited by their small size and consequent limitations on the amount of drug that can be loaded therein. It can also be difficult to obtain useful and controllable drug release kinetics from such fibers, and to control the placement and subsequent mobility of such fibers within the patient. The use of such fibers has therefore been limited.

### SUMMARY OF THE INVENTION

In one aspect, the present invention relates to drug-loaded fibers having high drug loading rates and that offer useful and controllable drug release kinetics.

In another aspect, the present invention relates to implants that comprise at least one drug-loaded fiber having a high drug loading rate and that offer useful and controllable drug release kinetics.

In another aspect, the present invention relates to methods of making drug-loaded fibers, and implants made therefrom, that have high drug loading rates and that offer useful and controllable drug release kinetics.

In yet another aspect, the present invention relates to methods of treating patients using the fibers of the present invention. The fibers of the present invention comprise a polymeric material and a drug. The fibers are characterized by a diameter of up to about 20 microns, and the drug located within the fibers is substantially in particulate form. In certain embodiments, the drug makes up at least about 20 weight percent of the fibers. The drug is either substantially insoluble in the polymer and solvent, or the amount of drug in the solution exceeds the solubility limit of the drug within either of the polymer or solvent. In some embodiments, the fibers of the present invention comprise an inner radial portion and an outer radial portion. A drug is located within the inner and/or outer radial portions.

The implants of the present invention are adapted for implantation into a patient's body. Embodiments of the implants of the present invention include one or more individual fibers, or other implant configurations made from one or more fibers such as yarns, ropes, tubes, and patches.

In one embodiment, the fibers of the present invention are made by a coaxial electrospinning process in which at least one solution is electrospun into a fiber. The solution includes a polymer, a solvent, and a drug. The drug is either substantially insoluble in the polymer and solvent, or the amount of drug in the solution exceeds the solubility limit of the drug within either of the polymer or solvent.

### BRIEF DESCRIPTION OF THE DRAWINGS

FIGS. 1a and 1b are schematic representations of side and cross-sectional views of a fiber, in accordance with an embodiment of the present invention.

FIGS. 2a and 2b are schematic representations of side and cross-sectional views side and cross-sectional views of a fiber, in accordance with an embodiment of the present invention.

FIG. 3a is a schematic representation of an electrospinning system used to manufacture fibers of the present invention.

FIG. 3b is a schematic representation of a co-axial needle (in cross-section) used in an electrospinning system of the present invention.

FIG. 4 is a schematic representation of an electrospinning system used to manufacture fibers of the present invention.

FIGS. 5a and 5b are scanning electron micrographs of a co-axial fiber having inner and outer radial portions, in accordance with an embodiment of the present invention.

FIG. 6 shows the drug release profile from certain fiber embodiments of the present invention.

FIG. 7 shows the drug release profile from certain fiber embodiments of the present invention.

FIG. 8 shows the drug release profile from certain fiber embodiments of the present invention.

FIG. 9 is a schematic representation of an electrospinning system used to manufacture yarns of the present invention.

FIGS. 10a, 10b, and 10c show yarns including the incorporation of radiopaque marker bands, in accordance with an embodiment of the present invention.

FIGS. 11a, 11b, and 11c show ropes of the present invention.

FIG. 12 is a schematic representation of a tube of the present invention.

FIG. 13 is a schematic representation of a patch of the present invention.

FIG. 14 shows ropes of the present invention successfully implanted into the epidural and intrathecal spaces of cadaveric dogs.

### DETAILED DESCRIPTION OF THE PREFERRED EMBODIMENTS

The present invention includes fibers, methods of making such fibers, implants made from such fibers, and methods of treating patients using such fibers. The inventors have found it possible to manufacture small fibers with surprisingly high drug loading rates, and drug release profiles that may be tailored to the specific requirements of numerous medical applications. In addition, the inventors are able to create various implant configurations from the fibers of the present invention to optimize desired drug delivery characteristics and to facilitate appropriate deliverability of the implant to the patient and subsequent implant mobility. As used herein, “drugs” and “therapeutic agents” are used synonymously to include small molecules, biologics, and other active agents used to produce a desired therapeutic effect.

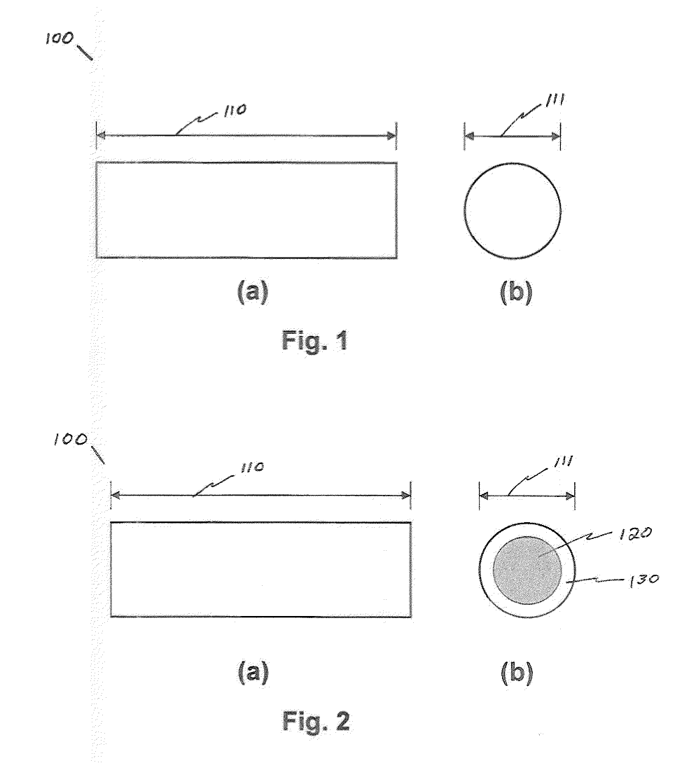

An example of a fiber of the present invention is shown schematically in FIGS. 1a and 1b. Fiber 100 is generally tubular in shape, and is characterized by a length 110 and a diameter 111. The fibers of the present invention are generally small enough to be useful for implantation to address a wide range of medical applications. As such, the fibers have a diameter 111 that is preferably up to about 20 microns. The length 110 of the fibers is dictated by the intended medical use, and generally may range from microns to millimeters to centimeters.

Fiber 100 is made from any suitable polymeric, biocompatible material and includes a drug embedded therein. Preferably, fiber 100 is made from a bioabsorbable material such that it degrades in a patient's body over time following implantation. The rate of degradation of the polymer material used to form the fiber 100 may be designed such that it either degrades following delivery of the drug therefrom, or as a means to control the drug delivery rate via the degradation process.

Examples of bioabsorbable materials that are useful in forming the fiber 100 of the present invention include: polyesters, such as poly(ε-caprolactone) (PCL), poly lactic-co-glycolic acid (PLGA), polyglycolic acid, poly(L-lactic acid), poly(DL-lactic acid); copolymers thereof such as poly(lactide-co-ε-caprolactone), poly(glycolide-co-ε-caprolactone), poly(lactide-co-glycolide), copolymers with polyethylene glycol (PEG); branched polyesters, such as poly(glycerol sebacate); poly(propylene fumarate); poly(ether esters) such as polydioxanone; poly(ortho esters); polyanhydrides such as poly(sebacic anhydride); polycarbonates such as poly(trimethylcarbonate) and related copolymers; polyhydroxyalkanoates such as 3-hydroxybutyrate, 3-hydroxyvalerate and related copolymers that may or may not be biologically derived; polyphosphazenes; poly(amino acids) such as poly (L-lysine), poly (glutamic acid) and related copolymers.

Examples of biologically derived bioabsorbable polymers that are useful in forming the fiber 100 of the present invention include: polypeptides such as collagen, elastin, albumin and gelatin; glycosaminoglycans such as hyaluronic acid, chondroitin sulfate, dermatan sulfate, keratan sulfate, heparan sulfate and heparin; chitosan and chitin; agarose; wheat gluten; polysaccharides such as starch, cellulose, pectin, dextran and dextran sulfate; and modified polysaccharides such as carboxymethylcellulose and cellulose acetate. Examples of other dissolvable or resorbable polymers include polyethylene glycol and poly(ethylene glycol-propylene glycol) copolymers that are known as pluronics and reverse pluronics.

Examples of non-biodegradable polymers that are useful in forming the fiber 100 of the present invention include: nylon4, 6; nylon 6; nylon 6,6; nylon 12; polyacrylic acid; polyacrylonitrile; poly(benzimidazole) (PBI); poly(etherimide) (PEI); poly(ethylenimine); poly(ethylene terephthalate); polystyrene; polysulfone; polyurethane; polyurethane urea; polyvinyl alcohol; poly(N-vinylcarbazole); polyvinyl chloride; poly (vinyl pyrrolidone); poly(vinylidene fluoride); poly(tetrafluoroethylene) (PTFE); polysiloxanes; and poly (methyl methacrylate).

In one embodiment as shown schematically in FIGS. 1a and 1b, fiber 100 is substantially homogeneous in composition such that it comprises a uniform polymer composition and drug dispersed substantially uniformly throughout. In a preferred embodiment, however, fiber 100 includes an inner radial portion 120 and outer radial portion 130, as shown in FIGS. 2a and 2b. The use of inner and outer radial portions 120, 130 allows for the tailoring of drug release kinetics. For example, in a preferred embodiment, substantially all of the drug within the fiber 100 is located within the inner radial portion 120 in its as-manufactured condition. In this preferred embodiment, the outer radial portion 130 is substantially free of drug in its as-manufactured condition, and may act as a drug diffusion barrier to control or limit the rate of drug delivery from the inner radial portion 120 into a patient following implantation of the fiber 100. In other embodiments, the outer radial portion 130 also includes a drug, which may be the same or different drug as contained in the inner radial portion 120. In yet other embodiments, substantially all of the drug within the fiber 100 is located within the outer radial portion 130, and the inner radial portion 120 is substantially free of drug in its as-manufactured condition. In a preferred embodiment of the invention, the fiber 100 includes inner and outer radial portions 120, 130 as shown in FIGS. 2a and 2b, the total diameter of the fiber is no more than about 20 microns, and the diameter of the outer radial portion is about 1-7 microns larger than the inner radial portion.

The amount of drug within the fibers of the present invention is preferably at least about 20 percent by weight. Using the methods of the present invention, the inventors have surprisingly found that high drug loading rates of 20 weight percent and higher (such as 25, 30, 35, 40, 45, 50 weight percent, and higher) are achievable. To achieve these high drug loading rates, drugs are used that are substantially insoluble in the polymer(s) of the fiber 100 (including any solvents used during the manufacturing process), or the amount of drug that is used is higher than the solubility limit of the drug in the polymer (or solvent). As such, and in contrast with known drug-loaded fiber technologies, the drug will not be dissolved within the polymer and associated solvents, but will exist in particulate form.

The fibers of the present invention are preferably manufactured using electrospinning techniques. Electrospinning is a process in which a continuous stream of polymer solution is ejected from a cylindrical tube or needle known as a “spinneret” towards a collection substrate by the application of both pressure and an electric field. During this process, the charge accumulation and evaporation of the solvent from the solution yields a single, long polymer fiber typically characterized by diameters from the nanometer to micron scale.

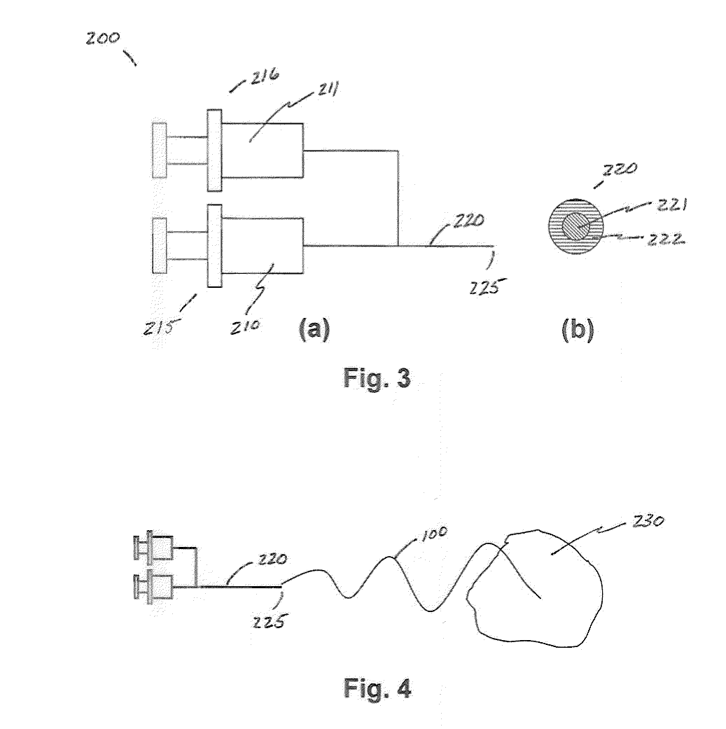

In a preferred embodiment, fibers of the present invention having inner and outer radial portions are manufactured using a co-axial spinneret system as schematically shown in FIG. 3. The system 200 includes an inner solution feed 210 and outer solution feed 211 loaded within respective syringes or similar containers 215, 216. The inner solution feed comprises a solution that includes a polymer, a solvent, and in a preferred embodiment, a therapeutic agent. As previously discussed, the drug is selected so as to be substantially insoluble in either of the polymer or solvent, or the amount of drug within the inner solution feed is selected to exceed the solubility limit of the drug within either of the polymer or solvent. The outer solution feed comprises a solution that includes a polymer and solvent, in a preferred embodiment. The syringes 215, 216 are preferably independently driven by one or more pumps that meters the rate of delivery of the solutions loaded therein. In this example, the spinneret 220 is a co-axial needle arrangement comprising an inner needle 221 in fluid communication with the inner solution feed 210, and an outer needle 222 in fluid communication with the outer solution feed 211. Preferably, the outer needle 222 comprises an electrically conductive material such as a suitable stainless steel, and more preferably, both the outer and inner needles 222, 221 comprise a conductive material. The co-axial needle arrangement of the spinneret 220, as shown in the cross-sectional view in FIG. 3a, results in the outer solution feed enveloping the inner solution feed.

As the inner and outer solution feeds move through the spinneret 220, they are charged by the application of an electric potential to the outer needle 222. The charge transfers through the outer needle 222 into the outer solution feed, and preferably into the inner solution feed. One or more grounded conductive substrates 230 are placed at a predetermined distance from the end 225 of the spinneret 220, preferably on the order of tens of centimeters, as shown in FIG. 4. The shape of the substrate(s) 230 is dictated by the form of implant desired to result from the electrospinning process. As the polymer solutions exit from the end 225 of the spinneret, the solvent(s) therein quickly evaporate, and the solutions are drawn into a small diameter fiber 100 through the action of electric forces such as charge repulsion and charge acceleration in the electric field formed between the spinneret 220 and grounded substrate(s) 230.

Although the electrospinning process is described with specific reference to a co-axial needle arrangement to produce a fiber 100 having inner and outer radial portions 120, 130, it should be appreciated that the present invention includes the formation of homogeneous fibers as described with reference to FIGS. 1a and 1b, in which a single feed solution is electrospun through a single needle spinneret.

The drugs used in the fibers of the present invention are any suitable drugs that are selected for treatment of the medical condition for which they are delivered, provided that they are either substantially insoluble in the polymers and solvents used in the fiber 100, or the amount of the drug exceeds the solubility limit of the drug in these materials. General categories of drugs that are useful in the present invention include, but are not limited to: opioids; ACE inhibitors; adenohypophoseal hormones; adrenergic neuron blocking agents; adrenocortical steroids; inhibitors of the biosynthesis of adrenocortical steroids; alpha-adrenergic agonists; alpha-adrenergic antagonists; selective alpha-two-adrenergic agonists; androgens; anti-addictive agents; antiandrogens; antiinfectives, such as antibiotics, antimicrobals, and antiviral agents; analgesics and analgesic combinations; anorexics; antihelminthics; antiarthritics; antiasthmatic agents; anticonvulsants; antidepressants; antidiabetic agents; antidiarrheals; antiemetic and prokinetic agents; antiepileptic agents; antiestrogens; antifungal agents; antihistamines; antiinflammatory agents; antimigraine preparations; antimuscarinic agents; antinauseants; antineoplastics; antiparasitic agents; antiparkinsonism drugs; antiplatelet agents; antiprogestins; antipruritics; antipsychotics; antipyretics; antispasmodics; anticholinergics; antithyroid agents; antitussives; azaspirodecanediones; sympathomimetics; xanthine derivatives; cardiovascular preparations, including potassium and calcium channel blockers, alpha blockers, beta blockers, and antiarrhythmics; antihypertensives; diuretics and antidiuretics; vasodilators, including general coronary, peripheral, and cerebral; central nervous system stimulants; vasoconstrictors; hormones, such as estradiol and other steroids, including corticosteroids; hypnotics; immunosuppressives; muscle relaxants; parasympatholytics; psychostimulants; sedatives; tranquilizers; nicotine and acid addition salts thereof; benzodiazepines; barbiturates; benzothiadiazides; beta-adrenergic agonists; beta-adrenergic antagonists; selective beta-one-adrenergic antagonists; selective beta-two-adrenergic antagonists; bile salts; agents affecting volume and composition of body fluids; butyrophenones; agents affecting calcification; catecholamines; cholinergic agonists; cholinesterase reactivators; dermatological agents; diphenylbutylpiperidines; ergot alkaloids; ganglionic blocking agents; hydantoins; agents for control of gastric acidity and treatment of peptic ulcers; hematopoietic agents; histamines; 5-hydroxytryptamine antagonists; drugs for the treatment of hyperlipiproteinemia; laxatives; methylxanthines; monoamine oxidase inhibitors; neuromuscular blocking agents; organic nitrates; pancreatic enzymes; phenothiazines; prostaglandins; retinoids; agents for spasticity and acute muscle spasms; succinimides; thioxanthines; thrombolytic agents; thyroid agents; inhibitors of tubular transport of organic compounds; drugs affecting uterine motility; anti-vasculogenesis and angiogenesis; vitamins; and the like; or a combination thereof.

Some embodiments of the invention comprise an active component that may include, but is not limited to: a) a corticosteroid, e.g., cortisone, hydrocortisone, prednisolone, beclomethasone propionate, dexamethasone, betamethasone, flumethasone, triamcinolone, triamcinolone acetonide, fluocinolone, fluocinolone acetonide, fluocinolone acetate, clobetasol propionate, or the like, or a combination thereof; b) an analgesic anti-inflammatory agent, e.g., acetaminophen, mefenamic acid, flufenamic acid, indomethacin, diclofenac, diclofenac sodium, alclofenac, ibufenac, oxyphenbutazone, phenylbutazone, ibuprofen, flurbiprofen, ketoprofen, salicylic acid, methylsalicylate, acetylsalicylic acid, 1-menthol, camphor, slindac, tolmetin sodium, naproxen, fenbufen, or the like, or a combination thereof; c) a hypnotic sedative, e.g., phenobarbital, amobarbital, cyclobarbital, lorazepam, haloperidol, or the like, or a combination thereof; d) a tranquilizer, e.g., fulphenazine, thioridazine, diazepam, flurazepam, chlorpromazine, or the like, or a combination thereof; e) an antihypertensive, e.g., clonidine, clonidine hydrochloride, bopinidol, timolol, pindolol, propranolol, propranolol hydrochloride, bupranolol, indenolol, bucumolol, nifedipine, bunitrolol, or the like, or a combination thereof; f) a hypotensive diuretic, e.g., bendroflumethiazide, polythiazide, methylchlorthiazide, trichlormethiazide, cyclopenthiazide, benzyl hydrochlorothiazide, hydrochlorothiazide, bumetanide, or the like, or a combination thereof; g) an antibiotic, e.g., penicillin, tetracycline, oxytetracycline, metacycline, doxycycline, minocycline, fradiomycin sulfate, erythromycin, chloramphenicol, or the like, or a combination thereof; h) an anesthetic, e.g., lydocaine, benzocaine, ethylaminobenzoate, or the like, or a combination thereof; i) another analgesic, e.g., acetylsalicylic acid, choline magnesium tri salicylate, acetaminophen, ibuprofen, fenoprofen, diflusinal, naproxen and the like; j) an antipruritic agent, e.g., bisabolol, oil of chamomile, chamazulene, allantoin, D-panthenol, glycyrrhetenic acid, a corticosteroid, an antihistamines and the like; k) an antimicrobial agent, e.g., methyl hydroxybenzoate, propyl hydroxybenzoate, chlorocresol, benzalkonium chlorides, nitrofurazone, nystatin, sulfacetamide, clotriamazole, or the like, or a combination thereof; l) an antifungal agent, e.g., pentamycin, amphotericin B, pyrrol nitrin, clotrimazole, or the like, or a combination thereof; m) a vitamin, e.g., vitamin A, ergocalciferol, cholecalciferol, octotriamine, riboflavin butyric acid ester, or the like, or a combination thereof; n) an antiepileptic, e.g., nitrazepam, meprobamate, clonazepam, or the like, or a combination thereof; o) an antihistamine, e.g., diphenhydramine hydrochloride, chlorpheniramine, diphenylimidazole, or the like, or a combination thereof; p) an antitussive, e.g., dextromethorphan, terbutaline, ephedrine, ephedrine hydrochloride, or the like, or a combination thereof; q) a sex hormone, e.g., progesterone, estradiol, estriol, estrone, or the like, or a combination thereof r) an antidepressant, e.g., doxepin; s) a vasodilator, e.g., nitroglycerin, isosorbide nitrate, nitroglycol, pentaerythritol tetranitrate, dipyridamole, or the like, or a combination thereof t) local anesthetics, e.g., procaine, benzocaine, chloroprocainc, cocaine, cyclomethycaine, dimethocaine/larocaine, propoxycaine, procaine/novocaine, proparacaine, tetracaine/amethocaine, lidocaine, articaine, bupivacaine, carticaine, cinchocaine/dibucaine, etidocaine, levobupivacaine, lidocaine/lignocaine, mepivacaine, piperocaine, prilocalne, ropivacaine, trimecaine, or the like; u) another drug, e.g., 5-fluorouracil, dihydroergotamine, desmopressin, digoxin, methoclopramide, domperidone, scopolamine, scopolamine hydrochloride, or the like, or a combination thereof or the like; or a combination thereof.

Any opioid can be used in the embodiments of the present invention. Useful opioids include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dihydromorphone, dihydroisomorphinc, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, heroin, hydrocodone, hydromorphone, hydromorphodone, hydroxypethidine, isomethadone, ketobcmidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, pantopon, papavcreturn, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tilidine, tramadol, pharmaceutically acceptable salts thereof and mixtures of any two or more thereof.

The fibers of the present invention may be used as individual implants that may be delivered within an injectable solution or as an implant without any associated solution. In other embodiments, the fibers are formed into other configurations, such as yarns, ropes, tubes, and patches. Such configurations are useful for a variety of medical applications and are used to yield desired drug delivery characteristics, deliverability, and mobility after delivery into a patient. For example, the fibers of the present invention are useful for injection into fluid-filled spaces within the body, such as joints, eye chambers, intrathecal spaces, and pericardial spaces. The fibers may also be injected or implanted into tissue, such as, for example, intramuscularly or subcutaneously, or placed into bodily lumens such as blood vessels. The fibers may also be formed into configurations that provide tissue anchoring characteristics. Examples of such configurations include ends that expand into T-Bar anchors, dart tipped or curved hooks, and the like. The fibers and implants of the present invention, and methods of making and using them, are further described with reference to the following non-limiting examples.

### Example 1

### Formation of Homogeneous Fibers

Homogeneous fibers made from poly e-caprolactone (PCL) and containing 10 wt % dexamethasone were manufactured in accordance with the present invention. A solution containing 15 wt % PCL in a chloroform and acetone solvent was placed in a syringe capped with an 18 gauge needle, and connected to a syringe pump set to deliver a flow rate of about 4 mL/h. A grounded mandrel coated with polytetrafluroroethylene was placed about 17 cm from the needle tip. An electric current was applied to the needle, and a fiber was electrospun according to the electrospinning technique as described herein. The fiber was characterized by a substantially homogeneous composition and morphology, and a diameter of about 10 microns, throughout which the dexamethasone was dispersed in particulate form.

### Example 2

### Formation of Core-Sheath Fibers

Fibers having inner and outer radial portions, or a so-called “core-sheath” structure,” were manufactured in accordance with the present invention.

A first set of core-sheath fibers were manufactured to have an outer radial portion comprising PCL and an inner radial portion comprising PCL and dexamethasone. These fibers were made by formulating an outer portion solution comprising 20 wt % PCL in chloroform/ethanol, and an inner portion solution comprising 20 wt % PCL in chloroform/acetone with 30 wt % (with respect to PCL) dexamethasone. A co-axial needle arrangement comprising a stainless steel outer tube having an inner diameter of about 2.3 mm, and a stainless steel inner tube having an outer diameter of about 0.9 mm and an inner diameter of about 0.6 mm, was used to deliver the outer and inner portion solutions, respectively, into an electrospinning process. The outer portion solution was delivered at a rate of about 23 mL/h, and the inner portion solution was delivered at a rate of about 12 mL/h. A grounded mandrel coated with polytetrafluroroethylene was placed about 20 cm from the needle tip. An electric current was applied to the needle, and a fiber was electrospun according to the electrospinning technique as described herein.

A second set of core-sheath fibers were manufactured to have an outer radial portion comprising poly lactic-co-glycolic acid (PLGA), and an inner radial portion comprising PCL and dexamethasone. These fibers were made by formulating an outer portion solution comprising 6 wt % PLGA in hexafluoroisopropanol, and an inner portion solution comprising 15 wt % PCL in chloroform/acetone with 30 wt % (with respect to PCL) dexamethasone. A co-axial needle arrangement comprising a stainless steel outer tube having an inner diameter of about 2.3 mm, and a stainless steel inner tube having an outer diameter of about 0.9 mm and an inner diameter of about 0.6 mm, was used to deliver the outer and inner portion solutions, respectively, into an electrospinning process. The outer portion solution was delivered at a rate of about 8 mL/h, and the inner portion solution was delivered at a rate of about 3 mL/h. An electric current was applied to the needle, and a fiber was electrospun according to the electrospinning technique as described herein.

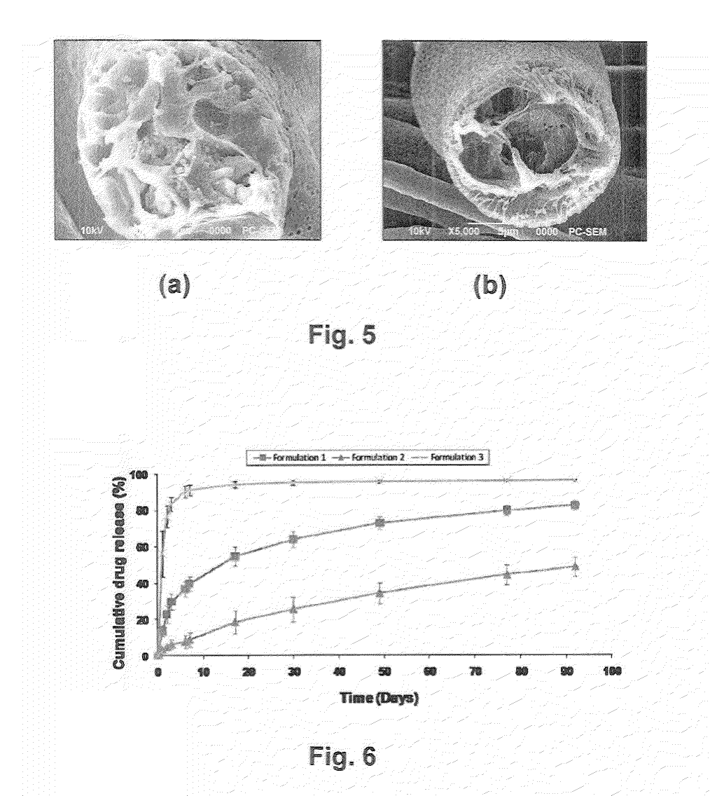

Both sets of core-sheath fibers were found to have a structure characterized by inner and outer radial portions. As shown in the scanning electron micrographs in FIGS. 5a and 5b, the fibers had average cross-sectional diameters between about 10 and 15 microns. As can be seen in FIG. 5a, the fibers produced in accordance with the present invention are characterized by morphology in which the drug exists in substantially particulate form because the amount of drug within the polymer solution used to make the fibers is insoluble in the polymer materials used, or exceeds the solubility limit of the drug within the polymer materials used. FIG. 5b shows the fiber shown in FIG. 5a after being soaked in methanol to extract the dexamethasone contained therein, thus revealing the remaining outer radial portion and showing that the inner radial portion was substantially comprised of dexamethasone.

### Example 3

### Comparison of Drug Release Rates

Fibers manufactured in accordance with Examples 1 and 2 were weighed and subsequently placed into a solution of phosphate buffered saline (PBS) and cyclodextrin. The rate of dexamethasone release from the fibers was measured using UV absorbance techniques. As expected, the homogeneous fiber structures manufactured in accordance with Example 1 resulted in a more pronounced “burst” drug release profile as compared to the core-sheath fiber structures manufactured in accordance with Example 2. As a result, the dexamethasone was found to be substantially released from the homogeneous fibers within about five hours. In comparison, the fibers in the first set of Example 2 yielded a dexamethasone release through about 120 hours after placement within the PBS solution, and the fibers in the second set of Example 2 yielded a dexamethasone release through about 170 hours after placement within the PBS solution.

### Example 4

### Tunability of Drug Release Rates Using Processing Conditions

Core-sheath fibers were manufactured having an outer radial portion comprising PLGA and an inner radial portion comprising PCL and dexamethasone. The fibers were made using an outer portion solution comprising 4 wt % PLGA in hexafluoroisopropanol, and an inner portion solution comprising 20 wt % PCL in chloroform/acetone with 20 wt % (with respect to the PCL) dexamethasone. The amount of dexamethasone within all fibers was about 13 wt %. A co-axial needle arrangement comprising a stainless steel outer tube having an inner diameter of about 2.3 mm, and a stainless steel inner tube having an outer diameter of about 0.9 mm and an inner diameter of about 0.6 mm, was used to deliver the outer and inner portion solutions, respectively, into an electrospinning process. A grounded mandrel coated with polytetrafluroroethylene was placed about 20 cm from the needle tip. An electric current was applied to the needle, and a fiber was electrospun according to the electrospinning technique as described herein. Three fiber structures were electrospun according to this Example, with only the feed rate of the inner and outer portion solutions being varied during the electrospinning process as follows:

| | Feed rate of | Feed rate of |
|---|---|---|
| | inner portion | outer portion |
| | solution (mL/h) | solution (mL/h) |
| Electrospinning process 1 | 3 | 8 |
| Electrospinning process 2 | 6 | 16 |
| Electrospinning process 3 | 9 | 24 |

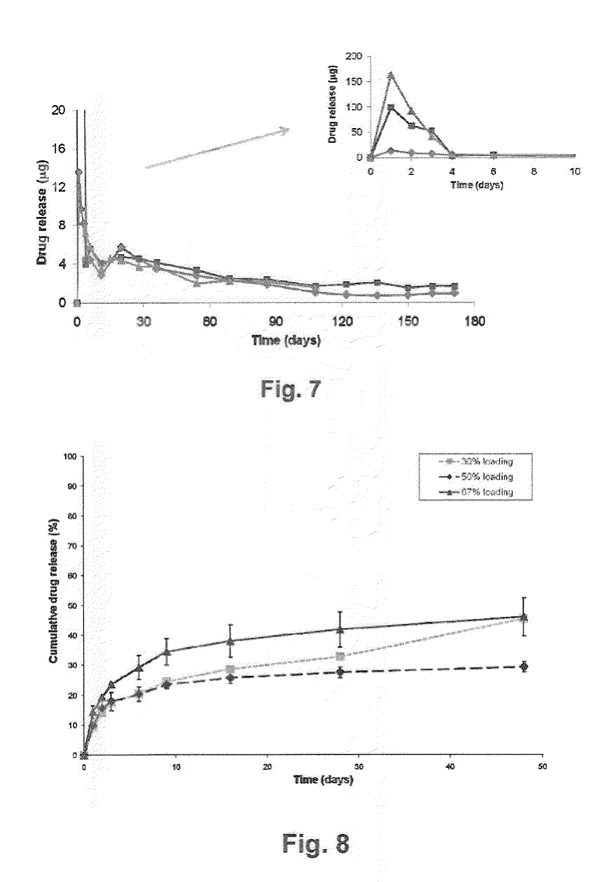

The fibers were weighed and subsequently placed into a solution of phosphate buffered saline (PBS) and cyclodextrin. The rate of dexamethasone release from the fibers was measured using UV absorbance techniques. The inventors surprisingly found that although both the dexamethasone loading and the relative PLGA to PCL ratio was substantially identical for all fibers, the elution profiles depended upon the feed rates of the inner and outer solutions during electrospinning. For example, as shown in FIG. 6, the use of the slowest feed rates for the inner and outer portion solutions resulted in a cumulative drug release that is slower than for fibers manufactured using higher solution feed rates. This is further demonstrated in FIG. 7, which shows that while the dexamethasone release from all fibers continued through at least about 110 days, the burst release resulting from fibers that were manufactured using the slowest feed rates for the inner and outer portion solutions was significantly lower compared to those fibers manufactured using higher solution feed rates, resulting in a more linear drug release profile.

### Example 5

### Fibers with High Drug Loading

Core-sheath fibers were manufactured to have an outer radial portion comprising poly lactic-co-glycolic acid (PLGA), and an inner radial portion comprising PCL and dexamethasone. These fibers were made from an outer portion solution comprising PLGA in chloroform and methanol, and an inner portion solution comprising PCL in chloroform and acetone. Three sets of fibers were manufactured using an electrospinning process analogous to the process described in Example 2—the core solution contained a high drug loading, 80 wt % with respect to PCL. By varying the outer solution conditions, three sets of fibers were produced: one set with a dexamethasone content of 30 wt %, a second set with a dexamethasone content of 50 wt %, and a third set with a dexamethasone content of 67 wt % (with respect to total fiber mass). The fibers were weighed and subsequently placed into a solution of phosphate buffered saline (PBS) and cyclodextrin. The rate of dexamethasone release from the fibers was measured using UV absorbance techniques. As shown in FIG. 8, the inventors have demonstrated that controlled drug release from fibers having high loading rates is achievable using the core-sheath structure of the fibers of the present invention.

### Example 6

### Formation of Yarns

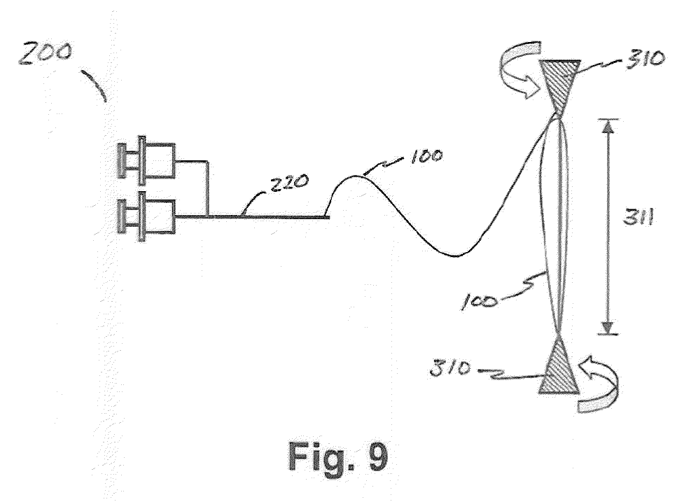

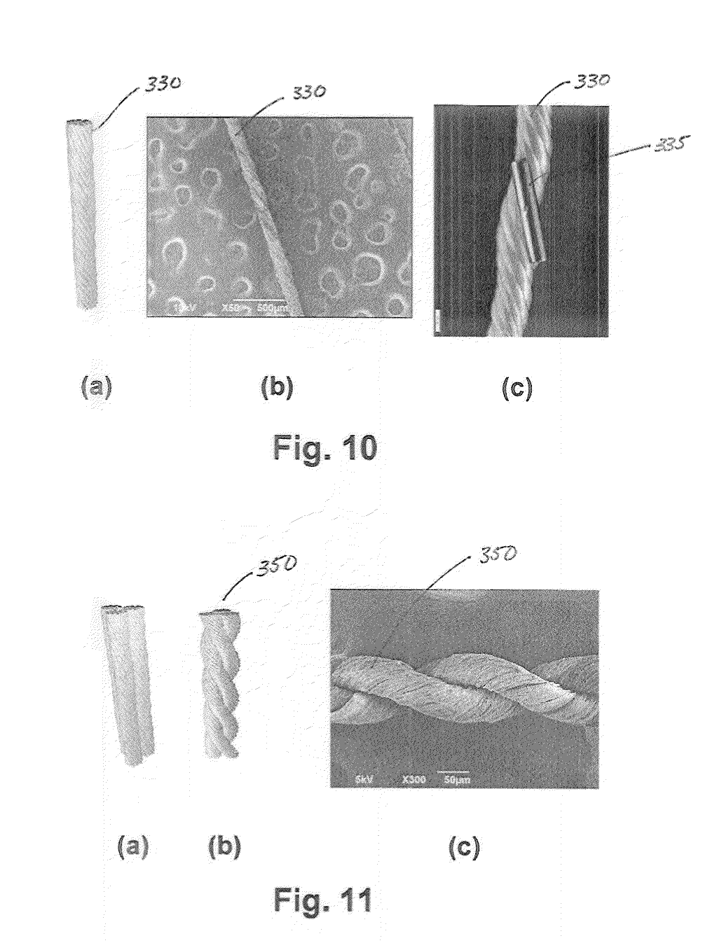

In one embodiment, fibers of the present invention are formed into drug-containing yarns. Such yarns are formed by electrospinning a fiber as previously described, with the fiber being collected on grounded collectors 310 that have a predetermined gap 311 there between, as shown in FIG. 9. During the electrospinning process, at least one fiber 100 is formed in the gap 311 between the collectors 310, which are rotated in opposite directions as the fiber(s) are deposited thereon. The result is a yarn structure 330 as schematically shown in FIG. 10a and as seen in the scanning electron micrograph of FIG. 10b, comprising aligned fiber(s) 100 in a twisted configuration. In a preferred embodiment, the fiber(s) have the core-sheath structure with inner and outer portions as previously described. After the formation of the yarn 330, it is cut or otherwise removed from the collectors 310 and used as-manufactured, or cut into smaller lengths, or sealed together with other yarn structures to make a long continuous yarn that may subsequently be used to create structures that are braided or knotted. The yarns of the present invention have an exemplary diameter in excess of 100 microns, a length of 1 millimeter or larger, and may optionally be manufactured large enough to include radiopaque marker bands 335 attached thereto, as shown in FIG. 10c.

### Example 7

### Formation of Ropes

In one embodiment, multiple yarns as described in Example 6 are made and twisted into a rope. As shown in FIG. 11a, such yarns are arranged parallel to each other and then twisted using any suitable mechanical means to form a rope, 350. The structure of a resulting exemplary is schematically shown in FIG. 11b and can be seen in the scanning electron micrograph of FIG. 11c. As one example, yarns as described in Example 6 were collected onto an electrospinning fixture consisting of two small diameter mandrels separated by a fixed distance. The yarns were twisted into a rope by rotating the mandrels at about 35 rpm in a counter-direction to each other for about 25-30 seconds.

In other embodiments, ropes according to the present invention comprise yarns with differing compositions, properties, drug release rates, and/or drugs loaded therein. For example, ropes of the present invention may be useful for applications in which two therapeutic agents work synergistically, which may be accomplished by forming one yarn comprising a first synergistic agent, forming another yarn comprising a second synergistic agent and/or an adjuvant to the first agent, and then twisting the yarns into a rope. As an example of such an application, agents such as bupivacaine and morphine may be loaded into individual yarns and subsequently formed into a rope.

The mechanical properties of the ropes of the present invention may be controlled by varying the number of yarns. For example, the inventors have measured the following mechanical properties of ropes made from PLGA yarns, where the number of yarns within the ropes varied between one, three, and six:

| | Young's | | Yield | Load at | Break | Break |
|---|---|---|---|---|---|---|
| | Modulus | Yield | Stress | Max. Stress | Strain | Stress |
| | (GPa) | Strain (%) | (MPa) | (N) | (%) | (MPa) |
| 1 yarn rope (n = 2) | 1.6 ± 0.1 | 1.6 ± 0.9 | 21 ± 14 | 1.3 ± 0.4 | 120 ± 10 | 50 ± 20 |
| 3 yarn rope (n = 2) | 1.3 ± 0.3 | 2.7 ± 0.01 | 27 ± 7 | 4.3 ± 0.5 | 120 ± 1 | 60 ± 10 |
| 6 yarn rope (n = 1) | 1.2 | 2.8 | 24 | 9.8 | 180 | 60 |

### Example 8

### Formation of Tubes

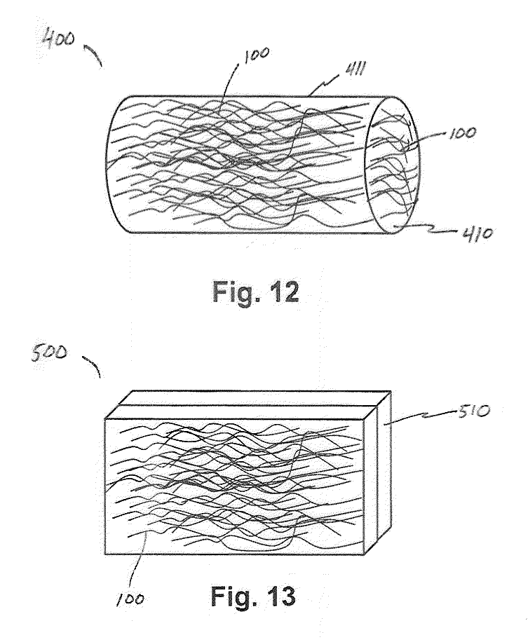

In one embodiment, fibers of the present invention are formed into drug-containing tubes. To make such tubes, drug-containing fibers are electrospun as previously described, but onto an elongated, grounded wire preferably having a diameter less than about 200 microns. After the solvent is evaporated following the electrospining process, the wire is extracted to yield a hollow tube 400 having a through cavity 410 and a side wall 411 that is made from one or more drug-containing fibers 100, as shown in FIG. 12. In a preferred embodiment, the tube 400 is cut into segments less than about 1 millimeter in length for implantation into a patient's body. Tubes with larger diameters, such as up to millimeters, and larger lengths, such as up to tens of millimeters or larger, may be made in accordance with the present invention for insertion into body lumens such a blood vessels for use as vascular grafts or the like.

In some embodiments, the tubes 400 of the present invention are further processed to include a drug inside the through cavity 410. This drug may be the same or different from the drug included in the fibers 100 that make up the side wall 411 of the tubes. In such embodiments, the inherent porosity of the side wall 411 can be altered using pressure, heat, or the application of solvent(s), which will in turn alter the delivery rate of drug from the fibers 100 of the tube side wall 411 and the through cavity 410. The use of drugs both within the fibers 100 and through cavities 410 allows for tailored drug delivery profiles such as an immediate burst release followed by a sustained release.

### Example 9

### Formation of Patches

In one embodiment, fibers of the present invention are formed into drug-containing patches 500, as shown in FIG. 13. Such patches are formed by electrospinning one or more fibers onto a metal substrate to create a sheet of fibers 100, which is then mechanically or chemically removed from the substrate and cut into a desired configuration. The subsequent fiber patch 500 may be further processed to tailor it for administration to a patient internally or externally. For example, the patch may include a polymeric coating layer 510 such as a hydrogel, absorbable polyesters such as those in the PLGA family of polymers, or polypeptides such as collagen, to help control the rate of drug delivery therefrom and/or to prevent tissue adhesion following implantation. In another example, the patch 500 includes a backing layer similar to the coating layer 510 that is used to attach the patch 500 to a patient's skin or internal bodily surfaces. Such patches are well-suited for wound healing applications because they may serve as scaffolds for cellular ingrowth and deliver therapeutic agents such as antibiotics. When designed to have a small mesh size, they may also act as physical barriers to pathogens while allowing fluid passage/drainage and nutrient transport.

In an alternate embodiment, a tube 400 made of drug-containing fibers is made from a patch 500 that is electrospun onto a grounded metal substrate. Following the electrospinning process, the patch is removed from the substrate and rolled into tubes, preferably having a diameter ranging from about 50 microns to about 1 millimeter.

### Example 10

### Treatment of Joint Conditions

In one embodiment, the fibers of the present invention are used to treat joint conditions such as osteoarthritis. Fibers may be delivered “dry” for this purpose, or may be included within a composition comprising a flowable material. If the latter, the flowable material is any suitable material that can be administered to an affected joint of a patient suffering from arthritis or other joint condition. Examples of such flowable materials are liquids such as saline, buffer, and isotonic solutions; gels such as those that include polymers such as alginates, glycosaminoglycans (GAGs), water soluble gums including agar, arabic, carob, carrageenans, cellulosics, chitin and chitosan based polymers, chondroitin sulfate, ethylene oxide containing polymers, poloxamers, ghatti, guars, hyaluronic acid, karaya, kadaya, locust bean, tragacanth, xantham, laminin, elastin, and other viscous media.

Fibers of the present invention having lengths on the order of hundreds of microns are suspended within the flowable material. The volume percent of the fibers within the flowable material is within any suitable range to provide for a desired therapeutic effect. The fibers are preferably biodegradable, and are made from suitable biocompatible materials that do not cause significant adverse effects when administered to a patient. Such materials include, but are not limited to synthetic absorbable polymers such as polyesters such as polydioxanone (PDO), polylactic acid (PLA), poly lactic-co-glycolic acid (PLGA), poly e-caprolactone (PCL) and copolymers thereof, poly glycolide (PGA), polyhydroxybutyrate (PUB), polyhydroxyalkanoate (PHA), poly glycerol sebacate (PGS); polycarbonates such as poly trimethylene carbonate (PTMC); polyanhydrides such as poly (sebacic anhydride), poly (bis carboxyphenoxypropane), degradable urethanes, and polyphasphazenes; natural polymers such as glycosaminoglycans, hyaluronic acid, laminin, elastin, collagen, gelatin, and albumin; and dissolvable polymers such as dextran, dextran sulfate, carboxymthyl cellulose, polyvinyl alcohol, polyethylene glycol and copolymers thereof, and pluronic polymers.

The fibers include inner and outer radial portions, as shown in FIGS. 2a and 2b. The inner portion includes a drug to treat arthritis and/or its symptoms, such as, for example, pain relievers such as bupivacaine, lidocaine, benzocaine, tetracaine, xylocaine, acetaminophen, para-aminosalicyclic acid, indomethacin, non-steroidal anti-inflammatory drugs (NSAIDs) such as diclofenac, ketoprofen, ibuprofen, naproxen, naproxcinoid, COX inhibitors including celecoxib, etoricoxib, lumiracoxib, meloxicam, nimesulfide, rofecoxib, valdecoxib, and opioids; antibiotics such as cyclines; biologics such as RNAi, oligonucleotides, proteins, and aptamers; antimicrobials such as chlorium dioxide and silver; MMP inhibitors; inhibitors of cytokines such as interleukin-1, interleukin 12, interleukin 23; tumor necrosis factor (TNF) and interferon gamma (IFN-γ); glucose derivatives such as aurothioglucose; and steroids such as cortisone, prednisone, and corticosteroids. In a preferred embodiment, the drug is an analgesic and the flowable material includes hyaluronic acid.

The fiber suspension within the flowable material is injected to affected joints using methods that are known in the art. In a preferred embodiment, the fiber suspension is directly injected via a needle injection into or near a joint to be treated, for example into the intra-articular space of the knee or the fat pad immediately adjacent to the knee joint capsule. As non-limiting examples, the compositions of the present invention are injectable into and around the joints of the knee, shoulder, hip, wrist, ankle, and hand, and are also injectable into the vertebral column, the mandible (jawbone), and sinus cavity. Injection can occur during or post-surgical procedures, or independently from surgical procedures. The advantages of the present invention can result in the minimization of the number of injections that are necessary to achieve a desired clinical effect.

### Example 11

### Treatment of Ocular Diseases

In another embodiment of the present invention, fibers are used to treat ocular diseases. Non-limiting examples of such diseases include scleritis, keratitis, corneal ulcers, corneal neovascularization, Fuchs' dystrophy, keratoconus, iritis, uveitis, cataracts, retinopathy, macular degeneration, macular edema, and glaucoma.

In a non-limiting example, co-axial fibers of the present invention are configured with outer and inner radial portions comprising PLGA and PCL, respectively, and fluocinolone acetonide contained within the inner radial portion. One or more fibers are cut into a lengths on the order of several millimeters, and injected into the vitreous humor of a patient's eye with a small diameter needle, in a procedure similar to intravitreal injection, to treat conditions such as diabetic macular edema, age-related macular degeneration, and/or posterior uveitis. Fluocinolone acetonide containing fibers may additionally be injected into the aqueous humor to treat anterior uveitis or scleritis.

### Example 12

### Treatment of Pain and CNS Disease

In another embodiment of the present invention, fibers are used to deliver drugs to the spine to treat pain (such as chronic pain, cancer pain, or other pain such as lower back pain) or diseases of the central nervous system (CNS) such as spasticity, Parkinson's, Alzheimer's, etc. For example, a rope of the present invention may be loaded with an appropriate therapeutic agent such as sufentanil, fentanyl, gentanyle, hydromorphone, morphine, bupivacaine, buprenorphine, or ziconitide, as described herein, and injected to a site near the pain receptors, such as the epidural or intrathecal space, for sustained pain relief with minimal systemic side effects.

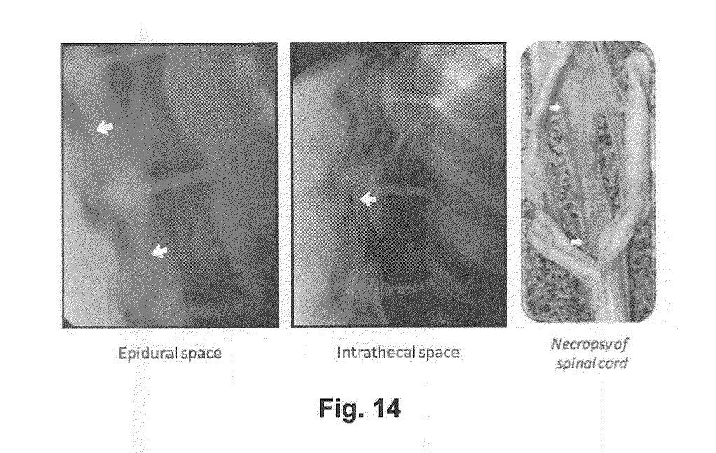

Ropes were manufactured and implanted into cadaveric dogs to demonstrate the applicability and deliverability of embodiments of the present invention. First, yarns were formed in accordance with Example 6. Radiopaque marker bands having an inner diameter larger than the yarn outer diameters were placed over one of the yarns. The yarns were adhered to fixtures and twisted into ropes, as described in Example 7. The ropes containing marker bands were implanted into a cadaveric dog using standard catheter-based delivery systems and methods. As shown in FIG. 14, ropes were successfully implanted into the epidural and intrathecal spaces.

### Example 13

### Systemic Delivery of Therapeutics Using Fibers

Fibers, either alone or in the form of tubes, yarns, or ropes of the present invention, may be injected into a patient for the systemic delivery of therapeutic agents. Such injections may be made as intramuscular or subcutaneous injections by a needle, either as dry fibers or as fibers in a flowable suspension. Such systemic delivery allows for sustained drug release for prolonged time periods up to several months. As one non-limiting example, risperidone is included within the inner portion of core-sheath fibers of the present invention, and administered by intramuscular injection for the treatment of schizophrenia.

The present invention includes fibers, methods of making such fibers, implants made from such fibers, and methods of treating patients using such fibers. The inventors have found it possible to manufacture small fibers with surprisingly high drug loading rates, and drug release profiles that may be tailored to the specific requirements of numerous medical applications. While aspects of the invention have been described with reference to example embodiments thereof, it will be understood by those skilled in the art that various changes in form and details may be made therein without departing from the scope of the invention.

We claim:

1. An implant for the delivery of a therapeutic agent to a location within a patient's body, comprising:
a fiber comprising a first polymeric material and having a diameter of up to about 20 microns, and
a first therapeutic agent within said fiber, wherein the amount of said therapeutic agent is greater than the solubility limit of said therapeutic agent in said first polymeric material.

2. The implant of claim 1, wherein said first therapeutic agent makes up at least about 20 weight percent of said fiber.

3. The implant of claim 1, wherein said first therapeutic agent makes up at least about 30 weight percent of said fiber.

4. The implant of claim 1, wherein said first therapeutic agent makes up at least about 40 weight percent of said fiber.

5. The implant of claim 1, wherein said fiber comprises an inner radial portion and an outer radial portion.

6. The implant of claim 5, wherein substantially all of said first therapeutic agent is located within said inner radial portion.

7. The implant of claim 6, further comprising a second therapeutic agent within said fiber.

8. The implant of claim 7, wherein substantially all of said second therapeutic agent is located within said outer radial portion.

9. The implant of claim 1, wherein said first polymeric material is bioabsorbable.

10. The implant of claim 5, further comprising a second polymeric material.

11. The implant of claim 10, wherein said inner radial portion comprises said first polymeric material and said outer radial portion comprises said second polymeric material, said first and second polymeric materials being different from one another.

12. The implant of claim 1, wherein said implant comprises at least one fiber formed into a yarn.

13. The implant of claim 12, wherein said implant comprises at least two yarns formed into a rope.

14. The implant of claim 13, wherein said at least two yarns each comprise a different therapeutic agent.

15. The implant of claim 13, further comprising a radiopaque band placed around at least one of said yarns.

16. The implant of claim 15, wherein said radiopaque band is bioabsorbable.

17. The implant of claim 1, wherein said implant comprises at least one fiber formed into a tube.

18. The implant of claim 1, wherein said implant comprises at least one fiber formed into a patch.

19. An implant for the delivery of a therapeutic agent to a location within a patient's body, comprising:
a fiber comprising a first polymeric material and having a diameter of up to about 20 microns, said fiber comprising an inner radial portion and an outer radial portion; and
a first therapeutic agent within said fiber, wherein substantially all of said first therapeutic agent is located within said inner radial portion, and the amount of said therapeutic agent is greater than the solubility limit of said therapeutic agent in said first polymeric material.

20. The implant of claim 20, wherein said first therapeutic agent makes up at least about 20 weight percent of said fiber.

21. The implant of claim 20, wherein said first therapeutic agent makes up at least about 30 weight percent of said fiber.

22. The implant of claim 20, wherein said first therapeutic agent makes up at least about 40 weight percent of said fiber.

23. The implant of claim 19, wherein said implant comprises at least one fiber formed into a yarn.

24. The implant of claim 20, wherein said implant comprises at least two yarns formed into a rope.

25. The implant of claim 24, wherein said at least two yarns each comprise a different therapeutic agent.

26. The implant of claim 24, further comprising a radiopaque band placed around at least one of said yarns.

27. The implant of claim 15, wherein said radiopaque band is bioabsorbable.

28. The implant of claim 19, wherein said implant comprises at least one fiber formed into a tube.

29. The implant of claim 19, wherein said implant comprises at least one fiber formed into a patch.
